(19) United States
(12) Patent Application Publication
(10) Pub. No.: US 2020/0300827 A1
(43) Pub. Date: Sep. 24, 2020

(54) GAS SENSING TATTOO STICKER

(71) Applicant: TAIWAN CARBON NANO TECHNOLOGY CORPORATION, MIAOLI COUNTY (TW)

(72) Inventors: Ching-Tung HSU, MIAOLI COUNTY (TW); Chun-Wei SHIH, MIAOLI COUNTY (TW); Kuang-Che LEE, MIAOLI COUNTY (TW); Chia-Hung LI, MIAOLI COUNTY (TW); Chien-Yao HUANG, MIAOLI COUNTY (TW); Chun-Hsien TSAI, MIAOLI COUNTY (TW); Ting-Chuan LEE, MIAOLI COUNTY (TW); Chun-Jung TSAI, MIAOLI COUNTY (TW)

(21) Appl. No.: 16/825,218

(22) Filed: Mar. 20, 2020

(30) Foreign Application Priority Data
Mar. 22, 2019 (TW) 108110104

(51) Int. Cl. G01N 33/00; A61B 5/08; G01N 33/497

(52) U.S. Cl. CPC G01N 33/0013; A61Q 1/025; G01N 33/497; A61B 5/082

(57) ABSTRACT

A gas-sensing tattoo sticker includes an adhesive layer, a coloring reaction layer, and a chemical reaction layer, disposed by stacking. The chemical reaction layer includes reaction zones capable of reacting with a gas to be tested to produce a chemical change; the coloring reaction layer includes coloring sides and correspondingly disposed reaction sides in contact with the reaction zones, and includes a coloring indicator to produce a coloring reaction corresponding to the chemical change of the reaction sides; to and the adhesive layer is provided on a side of the coloring reaction layer or the chemical reaction layer to provide adhesion, thereby completing the gas-sensing tattoo sticker, changes of gas in the surrounding environment can be sensed when air inlet sides are outwardly adhered on an object; and the smell of an object itself can be sensed when the air inlet sides are inwardly adhered on the object.

### FIELD OF THE INVENTION

The present invention relates to a sticker, and more particularly to a gas-sensing tattoo sticker used for gas sensing and having a light, thin and convenient structure.

### BACKGROUND OF THE INVENTION

In recent years, more and more studies have shown that the state of the human body can be detected through gaseous metabolism. For example, acetone can be detected in the exhaled gas of diabetic patients. Therefore, if such information can be recorded in real time, users can instantly know their own conditions.

Related techniques, such as Chinese patent publication no. CN108597621A, discloses a health status monitoring device, system, and method based on the theory of traditional Chinese medical science. In the patent, it is mentioned that a monitored user's smell information can be obtained through a smell scanner, and combined with other information such as facial image data, tongue texture image data, sound data, pulse wave data, etc.; thus, monitoring of the user's health status can be realized at low cost and without the full participation of Chinese medical personnel.

However, gas sensing devices such as the smell scanner described in the aforementioned patent are not only bulky, but also require power supply to operate normally, which interfere with the user's daily life and are not easy to operate, also it is hard to achieve the effect of monitoring anytime and anywhere, and the scope of application is relatively limited.

### SUMMARY OF THE INVENTION

An object of the present invention is to solve the drawbacks of the conventional gas sensing devices that are bulky and rely on continuous power supply to operate.

Another object of the present invention is to provide a gas-sensing device that is light, thin and convenient to use.

In order to achieve the above objects, the present invention provides a gas-sensing tattoo sticker including an adhesive layer, a coloring reaction layer, and a chemical reaction layer, which are disposed by stacking, wherein the chemical reaction layer includes at least one reaction zone capable of reacting with a gas to be tested to produce a chemical change, a side of the chemical reaction layer close to the gas to be tested is an air inlet side; the coloring reaction layer includes a coloring side and a reaction side opposite to each other, the reaction side contacts with the reaction zone of the chemical reaction layer; the coloring reaction layer includes a coloring indicator to produce a coloring reaction corresponding to the chemical change of the reaction side; and the adhesive layer has an area greater than or equal to that of the coloring reaction layer and the chemical reaction layer, and the adhesive layer is disposed on the coloring side of the coloring reaction layer, or a side of the chemical reaction layer away from the coloring reaction layer.

Accordingly, compared with the conventional gas sensing devices, the gas-sensing tattoo sticker of the present invention at least has the following advantages:

(1) The gas-sensing tattoo sticker of the present invention is capable of reacting with the gas to be tested through the reaction zones provided on the chemical reaction layer, and then undergoing the chemical change. The chemical change can show different colors through the reaction of the coloring indicator of the coloring reaction layer. Users can obtain the test results by directly observing the color change with the naked eye, or analyzing and interpreting the color change with an existing database, which is simpler and easier to use.

(2) When the air inlet side of the gas-sensing tattoo sticker is adhered outwardly on an object, changes of gas in the surrounding environment can be sensed; and when the air inlet side is adhered inwardly on an object, the smell of the object itself can be sensed. Compared with the conventional techniques, the gas-sensing tattoo sticker of the present invention is relatively extensive in applicability. For example, the gas-sensing tattoo sticker can be adhered on the thigh or groin of patients with reduced mobility to sense the micturition desire; when working in a potential toxic gas environment, the gas-sensing tattoo sticker can be adhered on the arm or the back of a hand to remind the user to pay attention to changes in the surrounding environment; and according to the documentation records, the exhaled gas of diabetics has a higher content of acetone, the patients with kidney disease have a higher content of ammonia when breathing, and such patients can be monitored for long or short period of time. In addition to the above-mentioned applications related to the human body, the gas-sensing tattoo sticker of the present invention can also be applied to animals and plants to monitor, for example, changes in color during the process of production, marketing, or growth.

### BRIEF DESCRIPTION OF THE DRAWINGS

FIG. 1 is a schematic diagram of a first usage mode of a gas-sensing tattoo sticker according to a first embodiment of the present invention;

FIG. 2 is a schematic diagram of a first usage mode of a gas-sensing tattoo sticker according to a second embodiment of the present invention;

FIG. 3 is a schematic diagram of a second usage mode of the gas-sensing tattoo sticker according to the first embodiment of the present invention;

FIG. 4 is a schematic diagram of a second usage mode of the gas-sensing tattoo sticker according to the second embodiment of the present invention;

FIG. 5 is a schematic diagram of applying the gas-sensing tattoo sticker of the present invention to a human body for sensing;

FIG. 6 is a schematic diagram of applying the gas-sensing tattoo sticker of the present invention to a plant for sensing;

FIG. 7 is a schematic diagram of a first usage mode of the gas-sensing tattoo sticker according to a third embodiment of the present invention;

FIG. 8 is a schematic diagram of a first usage mode of the gas-sensing tattoo sticker according to a fourth embodiment of the present invention; and

FIG. 9 is a schematic diagram of a first usage mode of the gas-sensing tattoo sticker according to a fifth embodiment of the present invention.

### DETAILED DESCRIPTION OF THE PREFERRED EMBODIMENTS

The detailed description and technical contents of the present invention are described below with reference to the drawings.

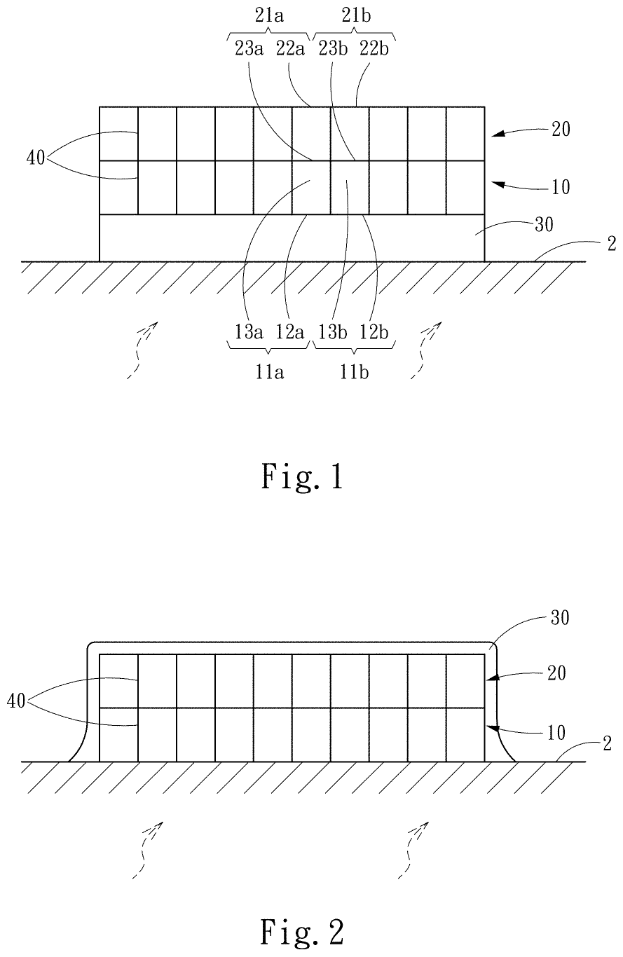

FIG. 1 is a schematic diagram of a first usage mode of a gas-sensing tattoo sticker according to a first embodiment of the present invention. The gas-sensing tattoo sticker mainly includes a chemical reaction layer 10, a coloring reaction layer 20 stacked with the chemical reaction layer 10, and an adhesive layer 30. The gas-sensing tattoo sticker further includes a partition portion 40.

The chemical reaction layer 10 is separated by the partition portion 40 to include a plurality of first areas 11a, 11b. The first areas 11a, 11b include reaction zones 13a, 13b respectively. The reaction zones 13a, 13b are capable of reacting with a gas to be tested (shown by arrows) to produce a chemical change. The reaction zones 13a, 13b can respectively include different kinds of chemicals, and can react with different target gases. For example, some of the reaction zones 13a, 13b can react with alkanes, some of the reaction zones 13a, 13b can react with alcohols, and some of the reaction zones 13a, 13b can react with sulfides. The partition portion 40 separates the adjacent first areas 11a, 11b, so that the reactions occurring in the adjacent first areas 11a, 11b do not affect each other. Wherein the chemical change can be a redox reaction, an acid-base reaction, an enzyme-catalytic reaction, a metal-catalytic reaction, a condensation reaction, a hydrolysis reaction, an addition reaction, an elimination reaction, a substitution reaction, or combinations thereof, but is not limited thereto. For a non-limiting example, an example of a redox reaction suitable for the present invention can be the oxidation of ethanol to acetaldehyde or acetic acid, an example of an enzyme-catalytic reaction can be glucose oxidase, and a metal catalyst can be a platinum catalyst.

In this way, assuming that the reaction zones 13a, 13b are coated with hydrazine (H₂N—NH₂), when a gas to be tested containing carbon dioxides reacts with the reaction zones 13a, 13b coated with hydrazine, carbazic acid (H₂NNHCOOH) will be produced, and color is developed using a redox indicator crystal violet.

If the reaction carried out in the reaction zones 13a, 13b is an irreversible reaction, the produced reaction results can be used as history information. The so-called history information refers to the recorded information relevant to all the adsorbed gas to be tested, that is, the history of the results is presented. However, if the reaction occurs in the reaction zones 13a, 13b includes gas adsorption and desorption, the reaction is a reversible reaction, which can be used as real-time information. The so-called real-time information refers to the current information, more specifically, some reactions will only last for a period of time, so the previous information is not recorded and only the current information is recorded. Therefore, at design stage, the diffusion coefficient can be appropriately adjusted to control the gas adsorption and desorption speeds, so that the chemical reactions in the reaction zones 13a, 13b are reversible reactions, that is, the history information and the real-time information can be recorded at the same time.

In the present invention, sides of the first areas 11a, 11b adjacent the gas to be tested are defined as air inlet sides 12a, 12b respectively. Furthermore, in one embodiment, a protective layer can be further disposed on the air inlet sides 12a, 12b to avoid interference or damage caused by gas directly entering the reaction zones 13a, 13b.

The coloring reaction layer 20 is also separated by the partition portion 40 to include a plurality of second areas 21a, 21b, the second areas 21a, 21b and the first areas 11a, 11b are stacked correspondingly to each other, and the second areas 21a, 21b include reaction sides 23a, 23b respectively, and coloring sides 22a, 22b respectively. The reaction sides 23a, 23b are in contact with the reaction zones 13a, 13b of the chemical reaction layer 10. The coloring sides 22a, 22b are disposed away from the reaction zones 13a, 13b, and the change of the colors can be observed through the coloring sides 22a, 22b.

Because the coloring reaction layer 20 includes a coloring indicator, when chemical changes are produced in the reaction zones 13a, 13b due to reactions, the coloring reaction layer 20 in contact with the reaction zones 13a, 13b will produce a coloring reaction corresponding to the chemical changes.

Wherein, composition of the coloring indicator is selected from a group consisting of a hydrate, a precipitate, a metal complex, and combinations thereof. Take the hydrate as an example, it can be dry cobaltous chloride which will become pink hydrate when meets water vapor; take the precipitate as an example, it can be black lead sulfide precipitate produced when lead acetate meets hydrogen sulfide; take the metal complex as an example, it can be oxygen coordinating and combining with iron ions in heme to present bright red color. The “coloring indicator” suitable for use in the present invention is not particularly limited. For example, the coloring indicator is further an acid-base indicator, a solvatochromism, or combinations thereof. It should be added to explain that the acid-base indicator suitable for use in the present invention is not particularly limited. For example, the acid-base indicator can be a colorimetric reagent such as bromothymol blue or phenolphthalein.

In this embodiment, the partition portion 40 is a partition wall that separates the adjacent first areas 11a, 11b and the adjacent second areas 21a, 21b to allow the gas to be tested to enter the air inlet side 12a to react with the reaction zone 13a without affecting the adjacent reaction zone 13b, and the reaction of the reaction zone 13a will only affect the reaction side 23a and the coloring side 22a, but will not affect the reaction side 23b and the coloring side 22b. In addition, in this embodiment, the chemical reaction layer 10 and the coloring reaction layer 20 are a double-layer structure independent of each other. However, in other embodiments, the chemical reaction layer 10 and the coloring reaction layer 20 can be one single-layer structure, that is, the chemical reaction layer 10 and the coloring reaction layer 20 are integrated into a single layer.

The adhesive layer 30 is disposed on a side of the chemical reaction layer 10 away from the coloring reaction layer 20; in this embodiment, the side refers to a side near the air inlet sides 12a, 12b. Since the adhesive layer 30 mainly provides adhesiveness for the gas-sensing tattoo sticker, the adhesive layer 30 can be made to have adhesiveness on only one side or both sides according to actual requirements; appropriate materials can also be selected according to the characteristics of an object or an individual 2 to be adhered, such as polyvinyl alcohol (PVA), but is not limited thereto.

In this embodiment, since the target gas to be tested is emitted from an object or the individual 2, the adhesive layer 30 is preferably air-permeable, so that the gas to be tested can pass through the adhesive layer 30 and enter the chemical reaction layer 10 through the air inlet sides 12a, 12b, and react with the reaction zones 13a, 13b in the chemical reaction layer 10.

Please continue to refer to FIG. 2, which is a schematic diagram of a gas-sensing tattoo sticker according to a second embodiment of the present invention. The same use situation as in the previous embodiment, the gas-sensing tattoo sticker is applied to sense a gas to be tested emitted from an object or the individual 2. However, compared with the first embodiment described above, the second embodiment is different only in the position where the adhesive layer 30 is disposed: in this embodiment, the adhesive layer 30 is disposed on the coloring sides 22a, 22b of the coloring reaction layer 20, and in order for the gas-sensing tattoo sticker to be firmly adhered to the object or the individual 2, an area of the adhesive layer 30 can be larger than an area of the coloring reaction layer 20 and an area of the chemical reaction layer 10.

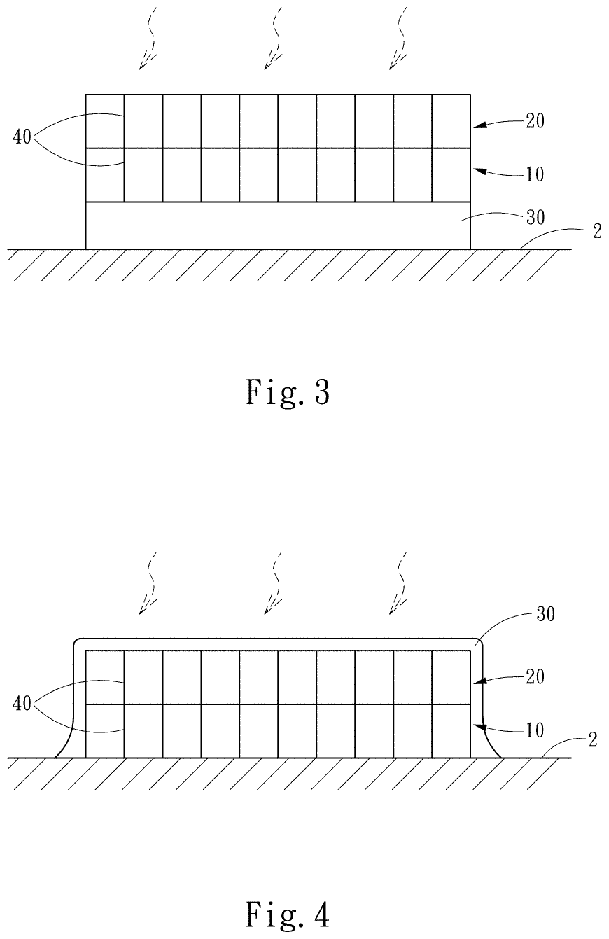

Please continue to refer to FIG. 3 and FIG. 4, which are respectively schematic diagrams of a second usage mode of the gas-sensing tattoo sticker according to the first embodiment and second embodiment of the present invention, that is, the gas-sensing tattoo sticker is adhered on the object or the individual 2 to sense changes of gas in the surrounding environment (as shown by the arrows). Compositions of the gas-sensing tattoo stickers shown in FIG. 3 and FIG. 4 are substantially the same as the foregoing, and will not be described in detail here. However, in these embodiments, in order to allow the gas to be tested to smoothly react with the reaction zones 13a, 13b, the coloring reaction layer 20 is preferably gas-permeable, so that the gas to be tested can pass through the coloring reaction layer 20 and enter the chemical reaction layer 10.

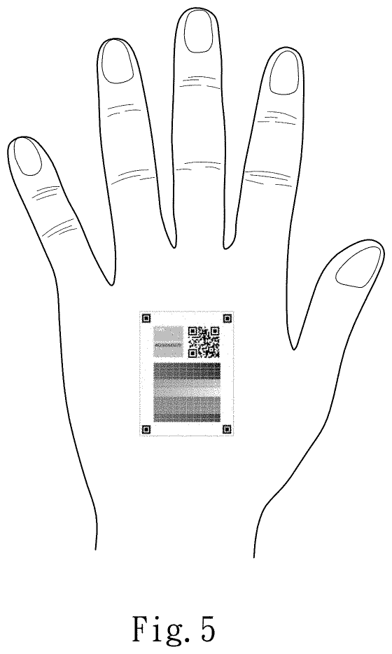

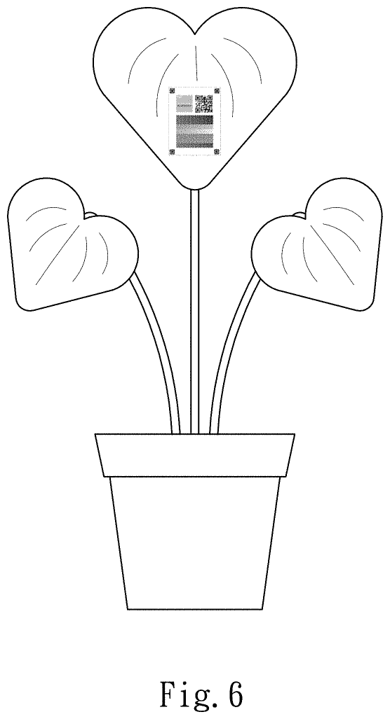

FIG. 5 and FIG. 6 are schematic diagrams of applying the gas-sensing tattoo sticker of the present invention to a human body and a plant respectively for sensing.

As shown in FIG. 5, users attach the gas-sensing tattoo sticker on the back of a hand which senses the metabolite smell or changes of smell emitted from the skin, and achieve the object of monitoring the body state by visual inspection or comparison with a database. Thus, the application areas include fast screening test and long-term monitoring of chronic diseases. In addition, different monitoring objects can be achieved depending on different locations of the adhesion, and the locations of the adhesion can be adjusted according to actual demands in usage. For example, if the urine smell of a patient needs to be monitored, the gas-sensing tattoo sticker of the present invention can be adhered on the thigh or groin; if the smell of excrements needs to be monitored, the gas-sensing tattoo sticker can be adhered to a position near a patient's hips; the gas-sensing tattoo sticker can also be adhered near the oral cavity to quickly monitor halitosis in daily life; alternatively, for users who are on exercise programs or dieting to lose weight, the gas-sensing tattoo sticker can also be adhered to appropriate parts of the body to monitor ketone bodies. Other applications can also achieve point-of-care testing (POCT) or diagnostic objects of medical clinics.

The gas-sensing tattoo sticker of the present invention can be used not only on a human body, but also can be adhered to a plant to monitor the smell the plant emits, as shown in FIG. 6. In a specific embodiment, fruits such as apples and bananas release ethylene during maturation. These reactions allow the gas-sensing tattoo sticker of the present invention to perform monitoring function, which is helpful to production and marketing and growth monitoring.

In addition to the structural modes shown in the foregoing first embodiment and second embodiment, the gas-sensing tattoo sticker of the present invention can further include other functional layers.

The following description is based on the structure of the second embodiment. However, in the first embodiment, functional layers which will be described below can also be similarly added without limitation.

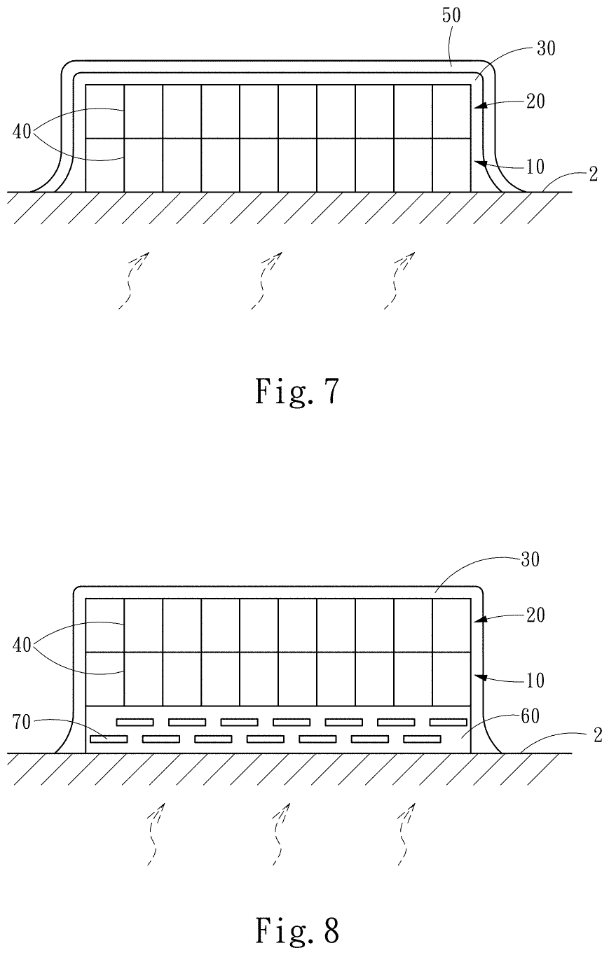

FIG. 7 is a schematic diagram of a gas-sensing tattoo sticker according to a third embodiment of the present invention. In this embodiment, an anti-reflection film 50 is further provided on an outermost side. The anti-reflection film 50 helps users to observe changes in color from the outside through an instrument or the naked eye and avoid interference.

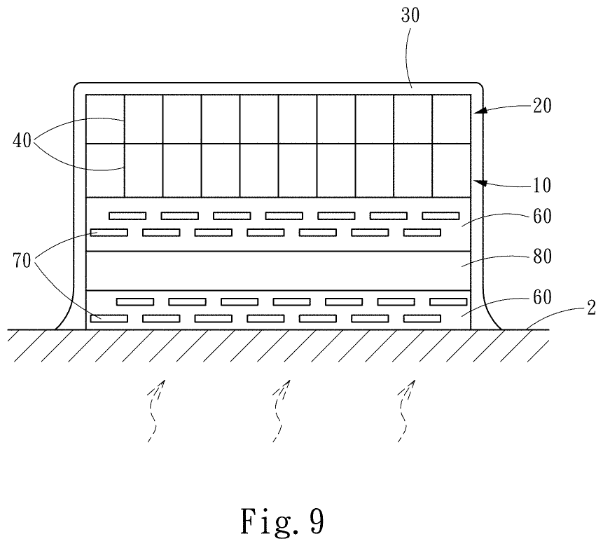

Please refer FIG. 8 and FIG. 9 along with FIG. 1, FIG. 8 and FIG. 9 are respectively schematic diagrams of a gas-sensing tattoo sticker according to a fourth embodiment and a fifth embodiment of the present invention. Compared with the structure of the second embodiment, the gas-sensing tattoo stickers of FIG. 8 and FIG. 9 are further provided with one layer or more than one layer of diffusion film 60 with gas screening function to achieve the effect of screening specific gases. The diffusion film 60 is disposed between a surface of the object or the individual 2 and the chemical reaction layer 10, that is, disposed close to the air inlet sides 12a, 12b.

In the case where the diffusion films 60 are provided, as shown in FIG. 9, the gases targeted by each of the diffusion films 60 can be different from each other. In addition, in order to adjust the diffusion path of gases in the diffusion films 60 to achieve changing the diffusion speeds of large and small molecules to obtain the effect of screening large and small molecules. In the embodiments of FIG. 8 and FIG. 9, each of the diffusion films 60 can be added with graphenes 70 of different sizes.

For more efficient adsorption of gas molecules, the gas-sensing tattoo sticker of the present invention can further include an adsorption molecule in the diffusion film 60 to achieve the above-mentioned object. The above-mentioned adsorption molecule can be any liquid, colloid, hole, or fiber film with an adsorption function. In a specific non-limiting example, glycerin can be used as the adsorption molecule; or in a specific non-limiting example, when holes are used as the adsorption molecule, characteristics of the holes are used to screen out larger-sized gas molecules. However, in the embodiment shown in FIG. 9, an adsorption layer 80 containing adsorption molecules can also be directly provided between a pair of the diffusion films 60, and such disposition can also obtain good adsorption effect.

It should be added to explain that even if the gas-sensing tattoo sticker of the present invention can be a structure formed by stacking the layers including the adhesive layer 30, there can be problem of poor water resistance. Therefore, in the various embodiments described in the foregoing, in order to reduce the interference of the external environment to the internal chemical reactions, an air-permeable film with water-blocking property can be optionally provided at an appropriate position near the air inlet sides 12a, 12b of the chemical reaction layer 10.

In order to facilitate the informationization of sensing data obtained each time, users can optionally have a one-dimensional bar code or a two-dimensional QR code designed on the gas-sensing tattoo sticker (as shown in FIG. 5 and FIG. 6); other methods, such as taking photographs to capture test results, and then analyzing and saving color changes with analysis software, can also achieve the object of informationization of sensing data. The monitoring data can be further classified into groups, and prediction and judgment are performed by AI machine learning.

In addition, the gas-sensing tattoo sticker of the present invention can be further provided with a plurality of colorimetric blocks, and the colorimetric blocks are arranged correspondingly to the reaction zones 13a, 13b. This design will help users to interpret color changes, and reduce identification errors.

Finally, in the above various embodiments, under the premise that the gas to be tested can enter the chemical reaction layer 10 and react with the reaction zones 13a, 13b, the disposing order of the chemical reaction layer 10, the coloring reaction layer 20, or the other functional layers can be exchanged with each other.

What is claimed is:

1. A gas-sensing tattoo sticker including an adhesive layer, a coloring reaction layer, and a chemical reaction layer, which are disposed by stacking, wherein:
the chemical reaction layer includes at least one reaction zone capable of reacting with a gas to be tested to produce a chemical change, a side of the chemical reaction layer close to the gas to be tested is an air inlet side;
the coloring reaction layer includes a coloring side and a reaction side opposite to each other, the reaction side contacts with the reaction zone of the chemical reaction layer; the coloring reaction layer includes a coloring indicator to produce a coloring reaction corresponding to the chemical change of the reaction side; and
the adhesive layer is disposed on the coloring side of the coloring reaction layer, or a side of the chemical reaction layer away from the coloring reaction layer.

2. The gas-sensing tattoo sticker as claimed in claim 1, wherein an anti-reflection film is further provided on an outermost side of the gas-sensing tattoo sticker.

3. The gas-sensing tattoo sticker as claimed in claim 1, wherein at least one diffusion film with gas screening function is disposed on a side close to the air inlet side.

4. The gas-sensing tattoo sticker as claimed in claim 3, wherein the diffusion film includes an adsorption molecule.

5. The gas-sensing tattoo sticker as claimed in claim 3, wherein the diffusion film further includes graphenes.

6. The gas-sensing tattoo sticker as claimed in claim 3, wherein a pair of diffusion films is disposed on a side near the air inlet side, and an adsorption layer is sandwiched between the pair of diffusion films.

7. The gas-sensing tattoo sticker as claimed in claim 1, wherein at least one diffusion film with gas screening function is further disposed on the air inlet side, and the at least one diffusion film with gas screening function is in direct contact with the air inlet side.

8. The gas-sensing tattoo sticker as claimed in claim 1, wherein the air inlet side is further provided with at least one film layer, and the film layer is selected from a group consisting of an adsorption layer, a diffusion film with gas screening function and combinations thereof.

9. The gas-sensing tattoo sticker as claimed in claim 1, wherein the coloring side is further provided with a colorimetric block.

10. The gas-sensing tattoo sticker as claimed in claim 1, wherein the adhesive layer has an area larger than that of the coloring reaction layer and the chemical reaction layer.

11. The gas-sensing tattoo sticker as claimed in claim 1, wherein the chemical change is a redox reaction, an acid-base reaction, an enzyme-catalytic reaction, a metal-catalytic reaction, a condensation reaction, a hydrolysis reaction, an addition reaction, an elimination reaction, a substitution reaction, or combinations thereof.

12. The gas-sensing tattoo sticker as claimed in claim 1, wherein the coloring indicator is an acid-base indicator, a solvatochromism, or combinations thereof.

13. The gas-sensing tattoo sticker as claimed in claim 1, wherein compositions of the coloring indicator are selected from a group consisting of a hydrate, a precipitate, a metal complex, and combinations thereof.
